(19) United States
(12) Patent Application Publication
(10) Pub. No.: US 2009/0036964 A1
(43) Pub. Date: Feb. 5, 2009

(54) Expandable Prostheses for Treating Atherosclerotic Lesions Including Vulnerable Plaques

(75) Inventors: James A. Heringes, Monmouth Junction, NJ (US); John Kula, Birdsboro, PA (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/184,055

(22) Filed: Jul. 31, 2008

Related U.S. Application Data
(60) Provisional application No. 60/953,440, filed on Aug. 1, 2007.
(60) Provisional application No. 60/970,947, filed on Sep. 8, 2007.
(60) Provisional application No. 60/977,199, filed on Oct. 3, 2007.
(60) Provisional application No. 61/050,229, filed on May 4, 2008.

(51) Int. Cl. A61F 2/92

(52) U.S. Cl. 623/1.2

(57) ABSTRACT

The invention provides expandable tubular prostheses that are designed for the treatment of atherosclerotic lesions, such as vulnerable plaques, and that are characterized by no foreshortening, optimal radial force and accuracy of deployment. In the treatment of vulnerable plaque, the device may be expanded in a blood vessel so that its central section at least partially contacts a vulnerable plaque lesion and/or the blood vessel wall in close proximity to the vulnerable plaque lesion. The invention also provides more general methods of treating atherosclerosis and promoting endothelialization using the prostheses of the invention.

### CROSS-REFERENCE TO RELATED APPLICATIONS

This application claims the benefit of each of U.S. Provisional Patent Application Ser. No. 61/050,229 filed May 4, 2008, 60/977,199 filed Oct. 3, 2007, 60/970,947 filed Sep. 8, 2007, and 60/953,440 filed Aug. 1, 2007, each of which is incorporated by reference herein in its entirety.

### FIELD OF THE INVENTION

The invention relates generally to the fields of expandable endoluminal vascular prostheses and their use in treating atherosclerotic lesions.

### BACKGROUND OF INVENTION

Vulnerable plaques, which are sometimes known as high-risk atherosclerotic plaques, include arterial atherosclerotic lesions characterized by a subluminal thrombotic lipid-rich pool of materials contained by and/or overlaid by a thin fibrous cap. Although vulnerable plaques are non-stenotic or marginally stenotic, it is believed that their rupture, resulting in the release of thrombotic contents, accounts for a significant portion of adverse cardiac events.

U.S. Publication No. 2002/0125799 discloses intravascular stents for the treatment of vulnerable plaque that consist of opposing end ring portions and a central strut portion having a zig-zag configuration that connects with the end portion at apices of the zig-zag structure, and is incorporated herein by reference in its entirety.

U.S. Publication No. 2005/0137678 discloses a low-profile resorbable polymer stent and compositions therefore, and is incorporated herein by reference in its entirety.

U.S. Publication No. 2005/0287184 discloses drug-delivery stent formulations for treating restenosis and vulnerable plaque, and is hereby incorporated by reference herein in its entirety.

### SUMMARY OF INVENTION

The present invention provides tubular endoluminal prostheses and methods for treating atherosclerotic lesions therewith. The prostheses of the invention have been designed to be particularly well suited to the treatment of vulnerable plaque lesions and for indications where the promotion of endothelialization is desired.

One embodiment of the invention provides an expandable tubular prosthesis in its unexpanded state that includes: at least one expandable at least substantially tubular portion disposed between two ends of the prosthesis, for example, a single central portion, that includes: a plurality of radially neighboring, longitudinally disposed sinusoidal members comprising peaks and troughs, the peaks and troughs of radially neighboring sinusoidal members being at least substantially in-phase with each other; and a plurality of radial connecting members that connect radially neighboring sinusoidal members, each radial connecting member having a first end and a second end and each including a plurality of segments joined by turns, wherein the first end and the second end of each radial connecting member connect to radially neighboring sinusoidal members at points of connection that are laterally offset, and wherein each radial connecting member comprises segments that at least substantially conform to the shape of the sinusoidal members to which the radial connecting member is connected.

Another embodiment of the invention provides an expandable tubular prosthesis in its unexpanded state that includes: at least one expandable at least substantially tubular portion disposed between two ends of the prosthesis, for example, a single central portion, that includes: a plurality of radially neighboring, longitudinally disposed sinusoidal members including peaks and troughs, the peaks and troughs of radially neighboring sinusoidal members being at least substantially in-phase with each other, and a plurality of radial connecting members that connect radially neighboring sinusoidal members, each radial connecting member having a first end and a second end and each including a plurality of segments joined by turns, wherein the first end and the second end of each radial connecting member connect to radially neighboring sinusoidal members at points of connection that are laterally offset, wherein each radial connecting member includes segments that at least substantially conform to the shape of the sinusoidal members to which the radial connecting member is connected, and wherein the sinusoidal members are connected at a plurality of lateral positions by a band of radial connecting members connecting radially neighboring sinusoidal members; and an end segment at each end of the prosthesis, each end segment including a laterally undulating member forming a radial band including apices, wherein the sinusoidal members connect to the end segments at or near the apices of each end segment.

A further embodiment of the invention provides a radially expandable tubular prosthesis in its unexpanded state that includes: at least one radially expandable at least substantially tubular portion disposed between two ends of the prosthesis, including: a plurality of radially neighboring, longitudinally disposed sinusoidal members comprising peaks and troughs, the peaks and troughs of radially neighboring sinusoidal members being at least substantially in-phase with each other, so that laterally adjacent, oppositely facing peaks of radially neighboring sinusoidal members are offset by 0.5 wavelength or about 0.5 wavelength with respect to the phase of the sinusoidal members, and wherein the width of the sinusoidal members narrows between at least substantially all of the peaks and troughs of the sinusoidal members; and a plurality of radial connecting members that connect radially neighboring sinusoidal members, each radial connecting member having a first end and a second end and each comprising a plurality of segments and turns, wherein the first end and the second end of each radial connecting member respectively connect to radially neighboring sinusoidal members at points of connection at or near the laterally adjacent peaks of the radially neighboring sinusoidal members, wherein each radial connecting member comprises segments that conform to the troughs of the sinusoidal members adjacent to the points of connection of the ends of the radial connecting member, said troughs being opposite the peaks of the radially neighboring sinusoidal members at or near which the ends of the radial connecting member are connected, and wherein each radial connecting member laterally extends beyond the points of connection of each end thereof at or near laterally adjacent peaks of radially neighboring sinusoidal members. In one variation, there is a single of said radially expandable at least substantially tubular portion disposed between two ends of the prosthesis. In another variation, there are at least two of said radially expandable at least substantially tubular portions disposed between two ends of the prosthesis.

Another embodiment of the invention provides a radially expandable tubular prosthesis in its unexpanded state that includes: at least one expandable at least substantially tubular portion disposed between two ends of the prosthesis, said portion composed of a super elastic metal alloy, having a wall thickness in the range of 40-100 microns, such as in the range of 40 to 70 microns, and comprising: a plurality of radially neighboring, longitudinally disposed sinusoidal members comprising peaks and troughs, the peaks and troughs of radially neighboring sinusoidal members being at least substantially in-phase with each other, so that laterally adjacent, oppositely facing peaks of radially neighboring sinusoidal members are offset with respect to the phase of the sinusoidal members; and a plurality of radial connecting members that connect radially neighboring sinusoidal members, each radial connecting member having a first end and a second end and each comprising a plurality of segments and turns, wherein the first end and the second end of each radial connecting member respectively connect to radially neighboring sinusoidal members at points of connection at or near the laterally adjacent peaks of the radially neighboring sinusoidal members, wherein each radial connecting member comprises segments that conform to the troughs of the sinusoidal members adjacent to the points of connection of the ends of the radial connecting member, said troughs being opposite the peaks of the radially neighboring sinusoidal members at or near which the ends of the radial connecting member are connected, and wherein each radial connecting member laterally extends beyond the points of connection of each end thereof at or near laterally adjacent peaks of radially neighboring sinusoidal members. In one variation, there is a single of said radially expandable at least substantially tubular portion disposed between two ends of the prosthesis. In another variation, there are at least two of said radially expandable at least substantially tubular portions disposed between two ends of the prosthesis.

A further embodiment of the invention provides a method for treating an atherosclerotic lesion, such as a vulnerable plaque, in a patient in need thereof, comprising the step of: deploying a prosthesis according to the invention at a site of an atherosclerotic lesion, such as a vulnerable plaque, in a blood vessel of a patient.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a partial section of an embodiment of an expandable prosthesis according to the invention.

FIG. 2 shows a partial section of an embodiment of an expandable prosthesis according to the invention.

FIG. 3 shows a partial section of an embodiment of an expandable prosthesis according to the invention.

FIG. 4 shows a partial section of an embodiment of an expandable prosthesis according to the invention.

FIG. 5 shows a partial section of an embodiment of an expandable prosthesis according to the invention.

FIG. 6 shows a partial section of an embodiment of an expandable prosthesis according to the invention.

FIG. 7 shows a partial section of an embodiment of an expandable prosthesis according to the invention.

FIGS. 8A and 8B show a partial section of an embodiment of an expandable prosthesis according to the invention.

FIG. 9A shows a partial section of an embodiment of an expandable prosthesis according to the invention.

FIG. 9B shows a radially expanded configuration of the embodiment of FIG. 9A

FIG. 9C shows various dimensions of a prosthesis according to the invention.

FIG. 10A shows a partial section of an embodiment of an expandable prosthesis that is variation of the embodiment of the invention shown in FIG. 9A.

FIG. 10B is an isometric view of the embodiment shown in FIG. 10A.

FIG. 10C is a photograph of a prosthesis having the design shown in FIGS. 10A and 10B, in its radially expanded state.

FIG. 11 shows the radial resistive and chronic outward forces profile of a 3.5 mm diameter version of a self-expanding prosthesis embodiment at different degrees of compression.

### DETAILED DESCRIPTION

The invention provides expandable tubular endovascular prostheses for the treatment of atherosclerotic lesions, including vulnerable plaques, and methods of treatment using the prostheses.

Various aspects of the invention are described below with reference to the appended figures.

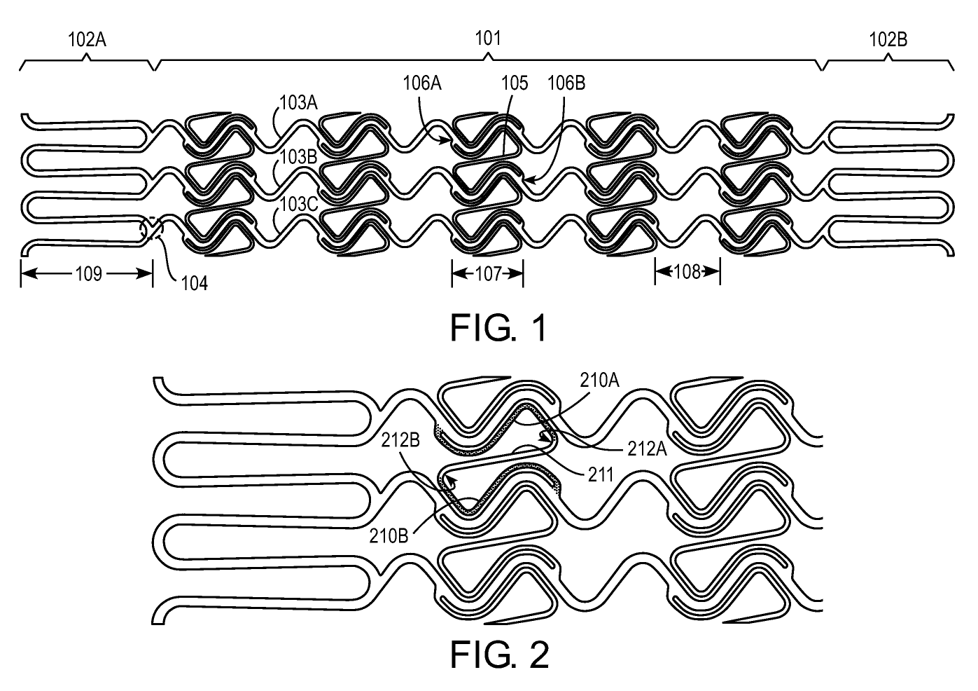

FIG. 1 shows an embodiment of the invention including a central section (central portion) 101 and two laterally undulating end sections 102A and B. The central section includes longitudinally disposed sinusoidal members (“backbone elements;” three shown: 103A-C) that are continuous and which connect at each end to the end sections at or near apices of the undulations of the end sections function 104). The peaks and troughs of the longitudinally disposed sinusoidal members are in-phase with each other. The use of in-phase backbone elements, versus out-of-phase backbone elements, prevents the torsions that would occur on expansion of the prosthesis if the sinusoidal backbone elements were not in-phase. At a plurality of lateral positions along the longitudinal axis of the prosthesis, radial connecting members that have two ends connect radially neighboring longitudinally disposed sinusoidal members (for example member 105 between connection points 106A and B). As shown, there are five bands of radial connecting members in the embodiment of the figure. The points of connection of a radial connecting member to radially adjacent longitudinally disposed sinusoidal members are laterally offset (compare points of connection 106A and B). In FIG. 1, the lateral offset (distance 107) is about 1.0 wavelength with respect to the phase of the longitudinally disposed sinusoidal members. Laterally neighboring bands of radial connecting elements are separated from each other (distance 108) by about 1.0 wavelength with respect to the phase of the longitudinally disposed sinusoidal members. The lateral width of the end sections (distance 109) is about 2.0 wavelengths with respect to the phase of the longitudinally disposed sinusoidal members in the embodiment of the figure. Other widths may also be used

FIG. 2 shows a close-up portion of one end of the section shown in FIG. 1. The radial connecting members include segments that are adjacent to the longitudinally disposed sinusoidal members to which they are connected and these segments conform to the sinusoidal shape of the longitudinally disposed sinusoidal members (see stippled segments 210A and B). A “center bar” segment 211 connects the segments 210A and B that conform to the sinusoidal members. The center bar in the embodiment of this figure is straight, but center bars characterized by curves are also provided by the invention. The areas 212A and B where the segment of the radial connecting element that conforms to a backbone element turns into the central bar of the radial connecting element is termed a “pivot loop.” In the embodiment of FIG. 1, the pivot loops are simple turns but they may also comprises additional and or more complex turns.

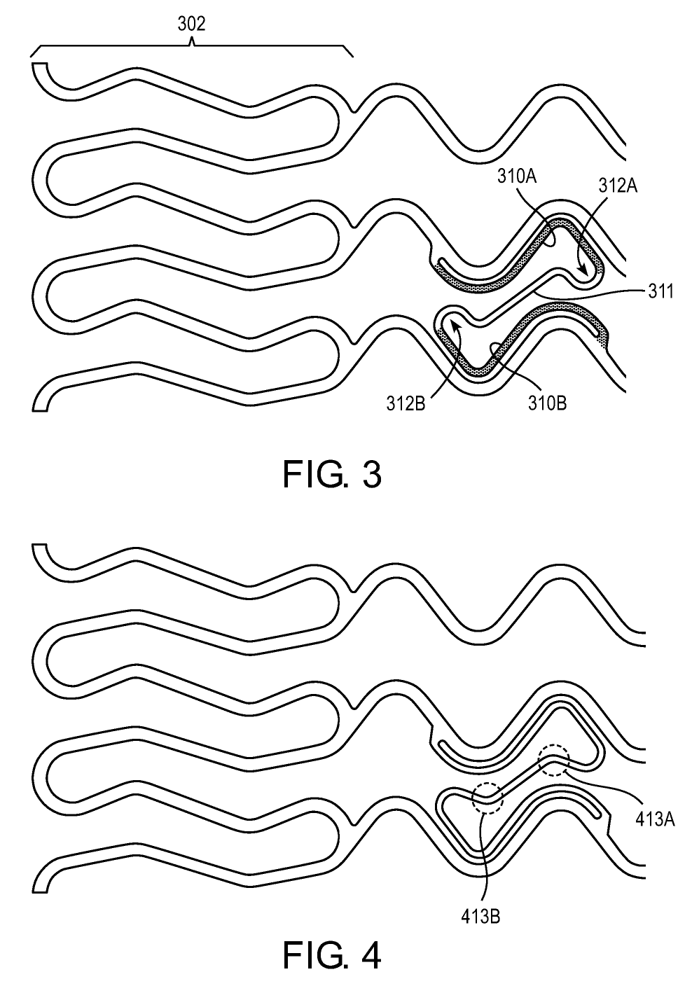

FIG. 3 shows an embodiment that is similar to that shown in FIG. 2, except that additional bends forming jagged undulations have been added to the lateral segments of the laterally undulating members of end section 302 of the prosthesis and the pivot loops 312A and B of the radial connecting members have been extended, as shown. Again, the radial connecting members are composed of segments 310A and B that conform to the shape of the sinusoidal members (delineated by stippling in figure) to which they are connected and a connecting bar segment 311 (no stippling) that connects to each of segments 310A and B. According to the terminology used herein, the pivot loops forming aspect of the radial connecting members are part of the center bar segment of the radial connecting members. Accordingly, in the embodiment shown, center bar 311 consists of a centrally disposed straight bar portion and a curved pivot loop portion at each end thereof. 312A and B respectively.

FIG. 4 shows a variation of the embodiment of FIG. 3, in which the angle of intersection between the pivot loop and the centrally disposed straight segment of the center bar at each of junctions 413A and B has been changed.

In FIGS. 1-4, the points of connection of the ends of the radial connecting to radially neighboring backbone elements occur between the peaks and troughs of the back bone elements.

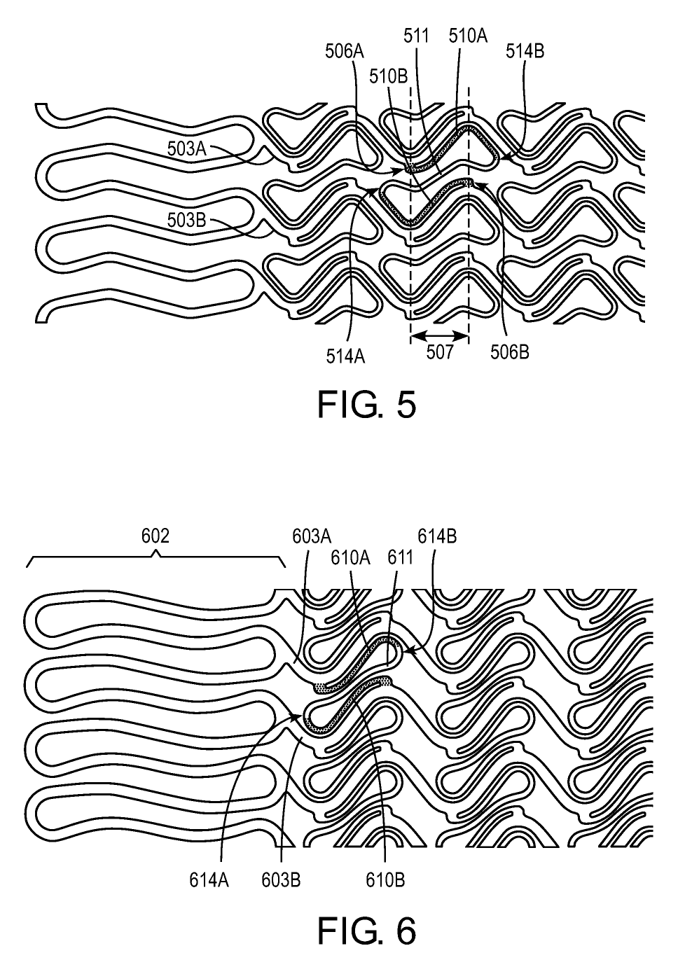

FIG. 5 shows an embodiment in which the shape of the radial connecting members is similar to that of the embodiment of FIG. 4, but in the embodiment of FIG. 5 the ends of the radial connecting members connect to the peaks of radially adjacent backbone elements and the points of connection of the ends of the radial connecting members, for example 506A and B, are laterally offset by 0.5 wavelength (distance 507) with respect to the phase of the backbone elements. As shown, in FIG. 5, every one of the peaks of the longitudinal sinusoidal members may be connected to a radial connecting member. One of the radial connecting members has been partially filled-in with stippling to indicate the segments 510A and B that conform to the sinusoidal members 503A and B to which the radial connecting member is attached. The respective center bar 511 including the pivot loops is shown as not stippled, between stippled segments 510A and B. Each of the conforming segments 510A and 510B includes a segment that proceeds from the point of connection at or near a peak of the sinusoidal member (in the direction of the peak that connects to the opposite end of the radial connecting member) down into the adjacent trough and turn to rise out of the trough whilst continuing to conform to the sinusoidal member before turning to become the pivot loop. The most laterally extended points 514A and B of the radial connecting member laterally extend beyond the points of connection of each end of the radial connecting member to the oppositely facing, laterally offset peaks of radially neighboring sinusoidal members 503A and B.

FIG. 6 shows an embodiment of the invention in which the bends of the laterally undulating segments of the end sections of the prosthesis and the bends of the radial connecting members are smoother and more rounded. The laterally oriented segments of end section 602 have a gentle sinusoidal curve. As shown for one of the radial connecting members, segments 610A and B thereof (stippled) conform to sinusoidal members (603A and B, respectively), and center bar 611 (not stippled) has a gentle sinusoidal curve along its length. Here also, it can be seen that the most laterally extended points of a radial connecting members (exemplified by 614A and B) laterally extend beyond the points of connection of each end of the radial connecting member to the oppositely facing, laterally offset, adjacent peaks of radially neighboring sinsusoidal members to which the radially connecting members connect.

As shown in FIGS. 1-6, the width of the backbone elements and the laterally undulating members of the end sections may be the same or about the same. As also shown in FIGS. 1-6, the width of segments of the radial connecting members may be less than that of the backbone elements, for example in the range of 30-60%, such as in the range of 30-40%, of the width of the backbone elements.

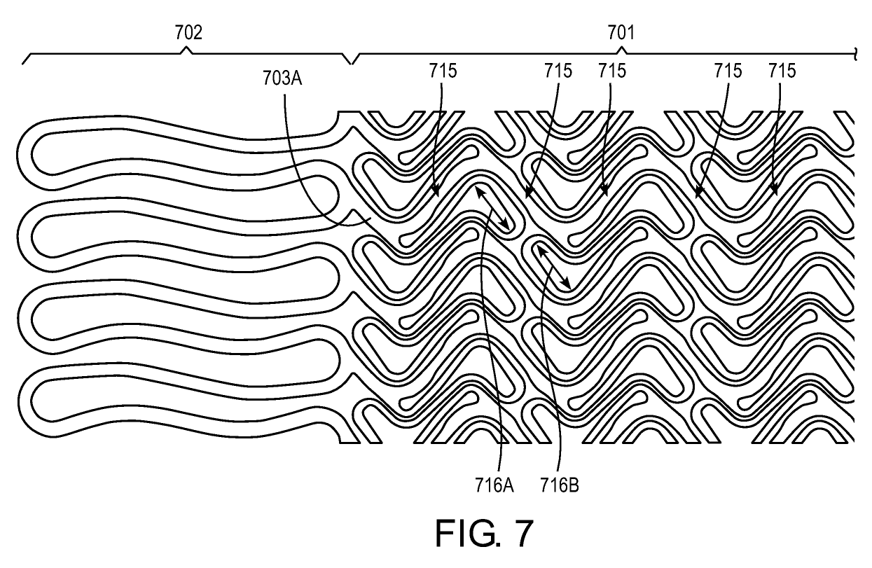

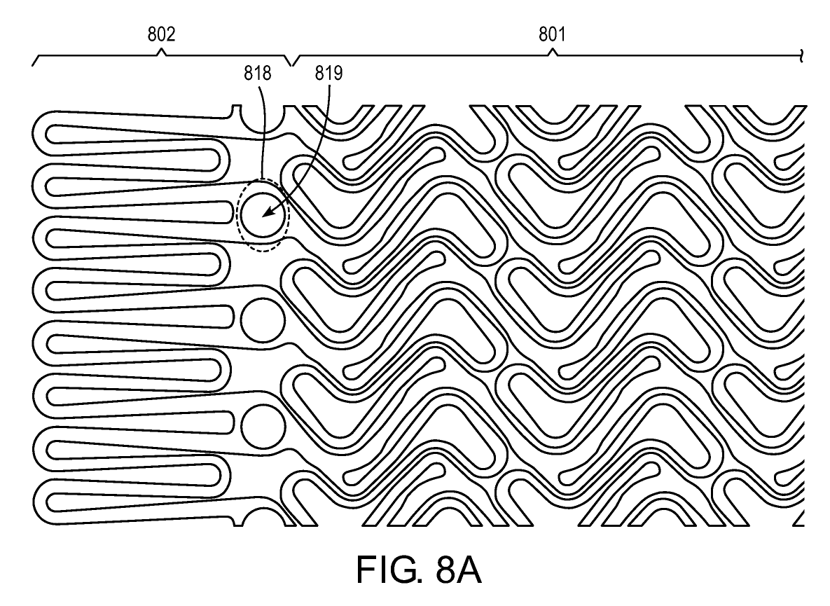

FIG. 7 shows an embodiment of the invention in which the regions between the peaks and troughs of the longitudinal sinusoidal backbone elements are narrowed (thinned) to increase overall flexibility of the device. For example, narrowings of width 715 are indicated between the peaks of the sinusoidal member 703A in the figure. This allows the prosthesis to more easily track around tight turns during delivery and be conformable upon expansion. As shown, in the central body portion of the prosthesis, each end of the radial connecting element protrudes from a peak of a radially neighboring lateral backbone element, the two peaks being laterally offset from one another. After protruding from the respective peaks, the radial connecting element proceeds in opposite lateral directions, with the segments that follow after the point of connection to a peak proceeding into and conforming with the side of the valley adjacent to the peak to which the end is connected and continuing to conform with the rising side of the valley. After rising from the valley (on both lateral ends of the radial connecting element), the element turns and conforms over the peak to which the opposite end is connected and over the start of the segment that protrudes from the opposite end to connect between the segments (halves) that initially proceeded into the valleys on radially adjacent sinusoidal backbone elements. Thus, as shown, a single radial connecting element of the embodiment laterally traverses two laterally adjacent offset peaks of radially neighboring backbone elements. As exemplified by dimensions 716A and B, the length of the segments of the radially connecting members that conform to the sinusoidal members while rising from the trough formed thereby is substantially increased so that the pivot loops of laterally neighboring radial connecting members are closely disposed when the prosthesis is in its unexpanded state. This increases the amount of coverage obtained between the sinusoidal members when the prosthesis is radially expanded.

It can also be seen in the embodiment of FIG. 7 that the most laterally extended points of a radial connecting members laterally extend beyond the points of connection of each end of the radial connecting member to the oppositely facing, laterally offset, adjacent peaks of radially neighboring sinsusoidal members to which the radially connecting members connect.

The central portion 701 of the prosthesis shown in FIG. 7 (partially shown; extending to end section at opposite end of prosthesis (not shown) is non-foreshortening.

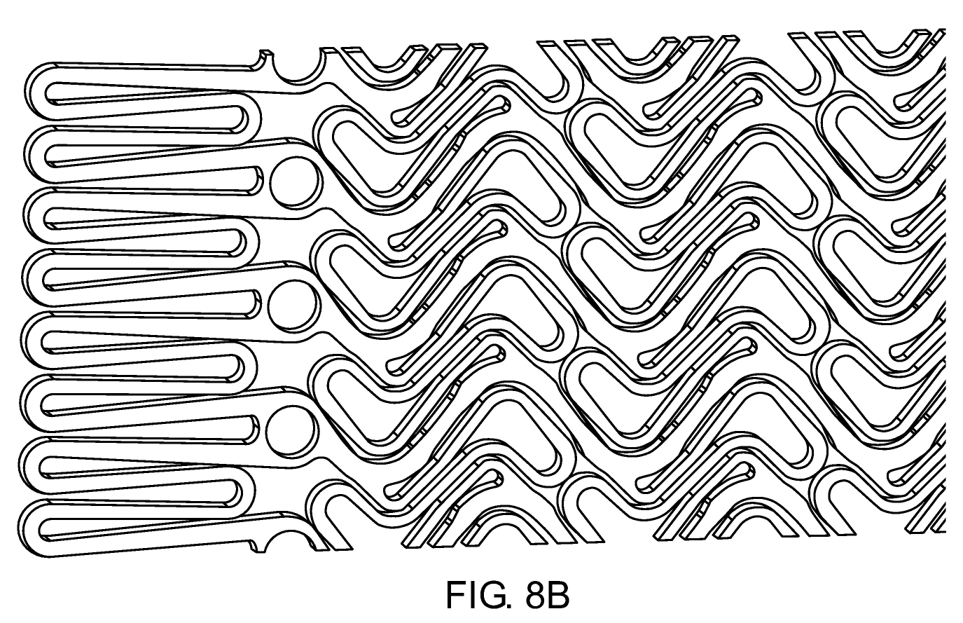

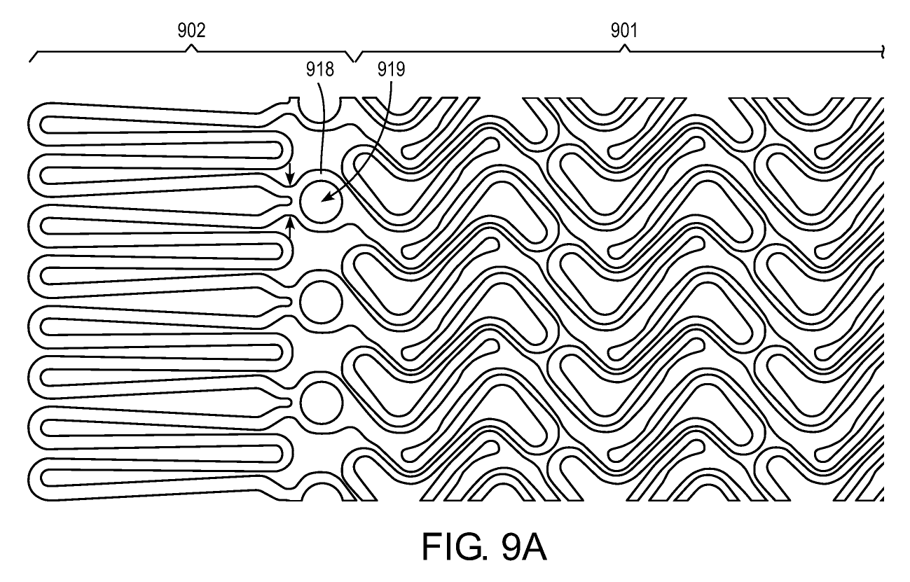

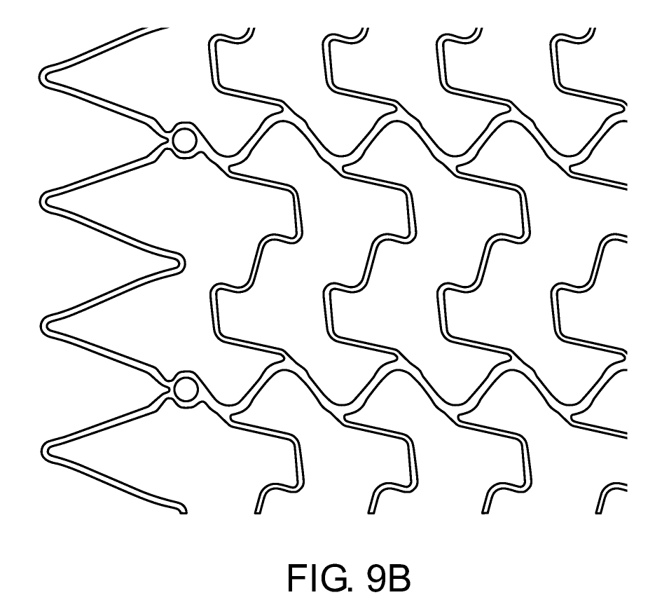

FIGS. 8A and B show an embodiment of the invention that includes improvements in the end sections of the prosthesis in comparison to the embodiment of FIG. 7. First, a greater number of turns in the undulating element of the end section between the radial positions of each lateral sinusoidal element has been added, resulting in more “end-points” being present in general for contacting the blood vessel and greater flexibility. Second, the elongate segments of the undulating element of the end section are tapered to reduce strains and improve flexibility. It has been found that these changes provide improved vessel contact and conformation of the end section to the vessel wall and minimize “tenting,” i.e., tautness of the vessel wall between the points of the prosthesis end section rather than conformation of the prosthesis end section to the shape of the vessel wall. Third, the structure connecting the lateral sinusoidal backbone elements with the undulating radial band of the end section has been changed so that the terminal end of a backbone element connects to two elongate segments of the undulating element (where an apex of a turn thereof would occur) by way of a section (exemplified by 818) that defines a hole 819, which is circular in the embodiment shown. One or more of the holes may be filled with radiopaque marker material, such as those known in the art, for example, gold or tantalum, to indicate the positions of the prosthesis during a procedure. The central portion or “working length” of the prosthesis is indicated by 801 and extends to the opposite end section of the prosthesis (not shown in the figure) which has the same structure as that shown. Radiopaque markers in structures 818 at each end of the prosthesis delineate the working length of the prosthesis and allow precise placement of the working area of the prosthesis at a location to be treated in a blood vessel. The central portion 801 of the prosthesis shown is non-foreshortening upon deployment.

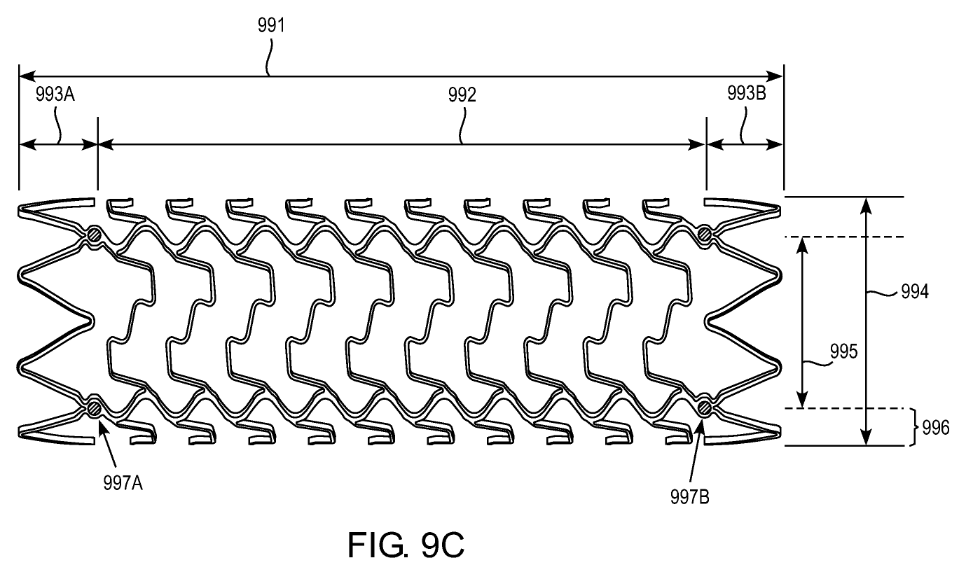

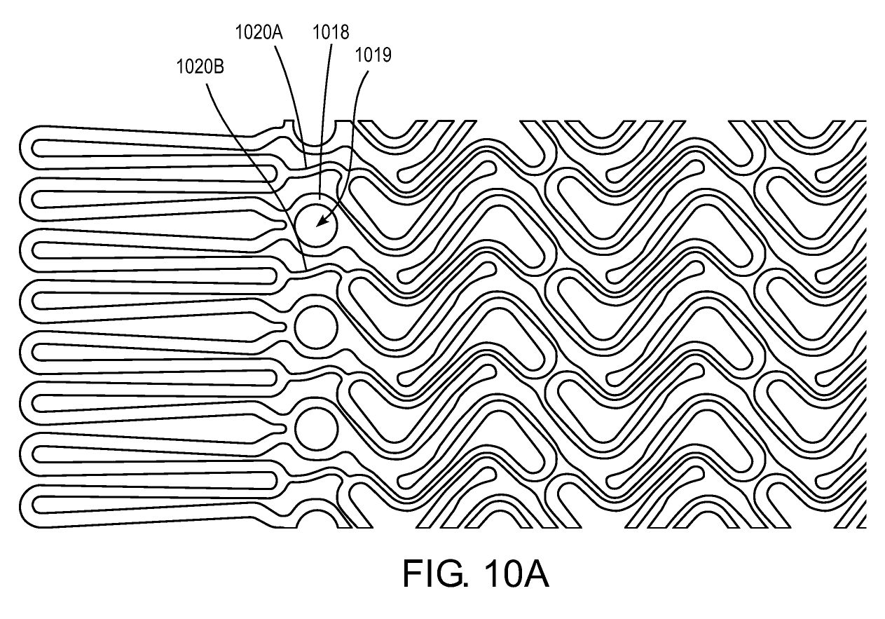

FIG. 9A shows a partial section of an embodiment of an expandable prosthesis according to the invention. The end section 902 of the embodiment of FIG. 9A is modified, versus that shown in FIG. 8, by the inclusion of structural hinges on the prosthesis end-facing side of the radiopaque marker containing/receiving hole 919 (formed by structure 918) and by the shape of the end section segments (struts) connecting to the structure forming the radiopaque marker containing/receiving hole. Two such hinge structure are indicated between the opposed arrows in FIG. 9A. Advantageously, inclusion of the hinges reduces strain that otherwise occurs about any inserted radiopaque marker material during radial expansion of the prosthesis and further reduces the foreshortening of the end sections of the prosthesis during radial expansion to about 1.0 to 1.5%. FIG. 9B shows a radially expanded configuration of the embodiment of FIG. 9A. As shown in FIGS. 9A and 9B, the hinge structure may be present adjacent to every radiopaque marker containing/receiving hole (irrespective of whether the hole contains radiopaque material). FIG. 9C illustrates various dimensions of the prosthesis (shown expanded). The total length of the prosthesis 991 is measured end-to-end. The working length of the prosthesis 992 is measured as the distance from the center of the radiopaque marker holding structure of one end of the prosthesis to the center of the radiopaque marker holding structure of the other end of the prosthesis. The length of the end sections, 993A and 993B, is measured from an end of the prosthesis to the center of the adjacent radiopaque marker holding structure of that end. Lengths 993A and 993B of the end sections may, for example, each be 1.6 mm or about 1.6 mm. Distance 994 is the unconstrained diameter of the expanded prosthesis. Distance 995 represents the “working” constrained (compressed) diameter of the prosthesis as may be experienced upon deployment in a blood vessel. The working range of compression 996 of the prosthesis (equal to ½ the difference between diameters 994 and 995 in the figure) may, for example, be in the range of 0.5 to 1.0 mm. It will be understood that in its working state of deployment in a blood vessel, the degree of radial compression may vary along the length of the prosthesis in accordance with the surrounding blood vessel and any matter therein. Arrows 997A and 997B each point to a radiopaque marker within a radiopaque marker holding structure, at opposite ends of the prosthesis respectively. In one 3.5 mm unconstrained diameter embodiment of the prosthesis, each end of the prosthesis has four radiopaque markers (as in FIG. 9C, where two of the four are shown at each end). In one 4.0 mm unconstrained diameter embodiment of the prosthesis, each end of the prosthesis has five radiopaque markers.

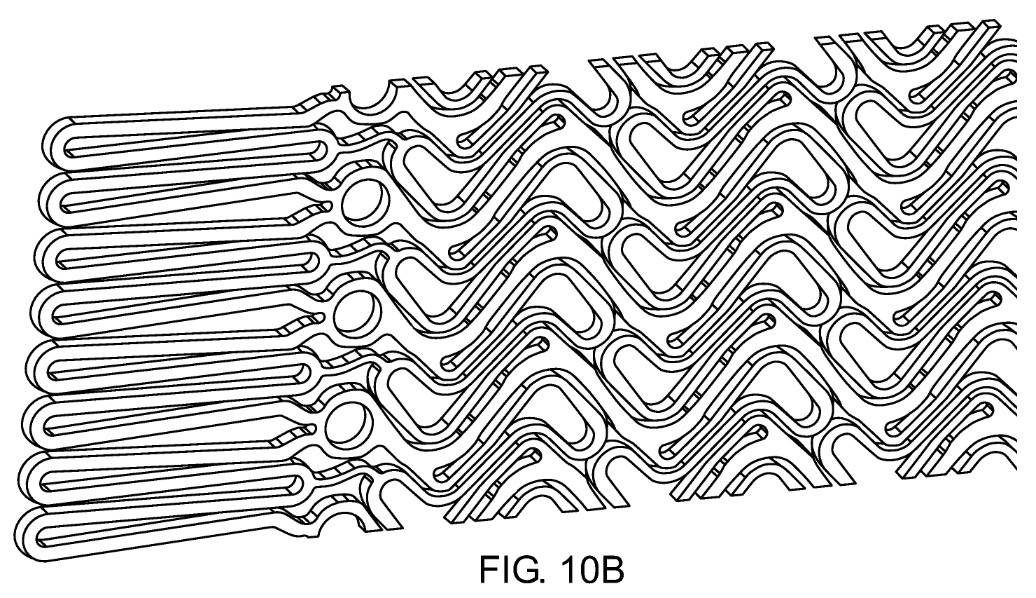

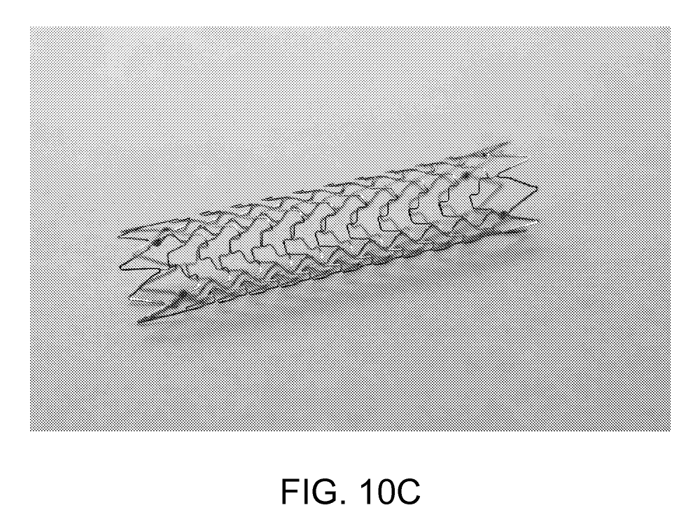

FIGS. 10A and B show a partial section (“flattened” view) of an embodiment of an expandable prosthesis that is variation of the embodiment of the invention shown in FIG. 9A. Specifically, the embodiment shown in FIGS. 10A and B further includes links (exemplified by members 1020A and B in FIG. 10A) that connect the internal apices of the sinuate member (that forms an end segment of the prosthesis) that lay between the radiopaque marker containing/receiving structures to the laterally neighboring apices (turn) of the laterally neighboring radial connecting members. The links may be provided at every position (as shown) or at one or more of the possible positions, for example, in an alternating manner. These “bridge” links facilitate loading of the prosthesis onto the delivery catheter by preventing edges from catching and also encourage even/controlled expansion of the end segments of the prosthesis upon deployment. As shown, each of the links is slightly sinuate. FIG. 10B is an isometric view of the embodiment shown in FIG. 10A. FIG. 10C is a photograph of a prosthesis having the design shown in FIGS. 10A and 10B, in its radially expanded state.

The wall thickness, longitudinal backbone element, and radial connecting members of prostheses of the invention such as those shown in FIGS. 7-10 may, for example, have the dimensions shown below in the Table 1. The percent of metal area (versus open area) may be in the range of 13-16%.

TABLE 1

| Parameter | Range (inches) | Range (microns) |
|---|---|---|
| Wall thickness | 0.00225 | 56 |
| Backbone width | 0.003-0.004 | 75-100 |
| Radial connection member width | 0.0015-0.0031 | 38-78 |

The prostheses of the invention may be provided in different total (overall) lengths such as 10 mm, 15 mm, 20 mm, 25 mm and 30 mm. Thus, in one embodiment, the total length of a prosthesis according to the invention may be in the range of 10-30 mm, for example, in the range of 15-25 mm. The longitudinal length of each end section of a prosthesis according to the invention may be in the range of 1.4 mm-2.0 mm, such as equal to or about 1.6 mm. The unconstrained expanded radial diameter of a prosthesis of the invention may, for example, be 3.5 mm, 4.0 mm, or 4.5 mm. In one embodiment, the radial diameter of a prosthesis according to the invention in an expanded, unconstrained state is in the range of 3.0 mm to 5.0 mm, such as in the range of 3.5 mm-4.5 mm. A prosthesis according to the invention in its expanded working state may, for example, have a working range of radial compression of 0.5 mm-1.0 mm.

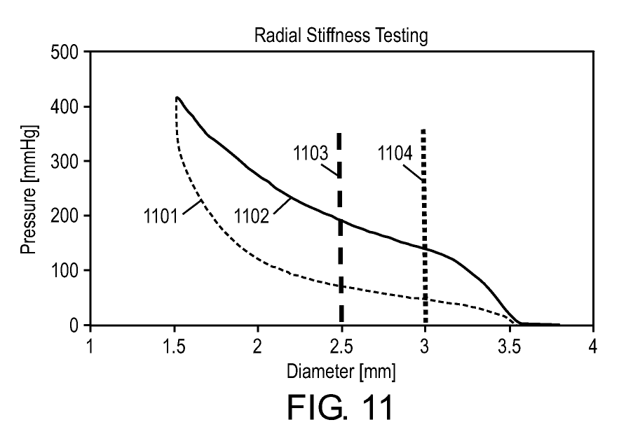

FIG. 11 shows the radial resistive and chronic outward forces profile of a 3.5 mm diameter version of a self-expanding prosthesis embodiment like that in FIG. 9C at different degrees of radial compression. The chronic outward force 1101 is the force the prosthesis exerts on the vessel wall (and any plaque/lesion), i.e., it is the dilation force exerted. The radial resistive force 1102 is the force the prosthesis exerts to resist the recoil of the plaque and vessel wall, i.e., it is the crush resistance force. The preferred working range of compression is between lower limit 1103 and upper limit 1104. Thus, advantageously, self-expanding prostheses according the invention in their working state are compressed by the surrounding vessel wall while simultaneously exerting a chronic outward force against the vessel wall.

Advantageously, the expandable prostheses of the invention are characterized by minimal/no foreshortening, optimized radial forces and accuracy of deployment. It is believed that no currently available self-expanding stent or self-expanding endovascular prosthesis provides this combination of features.

In view of the above and without limitation, the following embodiments are also provided by the invention.

One embodiment of the invention provides an expandable tubular prosthesis that includes: an expandable at least substantially tubular central portion disposed between two ends of the prosthesis, comprising; a plurality of radially neighboring, longitudinally disposed sinusoidal members comprising peaks and troughs, the peaks and troughs of radially neighboring sinusoidal members being at least substantially in-phase with each other; and a plurality of radial connecting members that connect radially neighboring sinusoidal members, each radial connecting member having a first end and a second end and each comprising a plurality of segments joined by turns, wherein the first end and the second end of each radial connecting member connect to radially neighboring sinusoidal members at points of connection that are laterally offset, and wherein each radial connecting member comprises segments that at least substantially conform to the shape of the sinusoidal members to which the radial connecting member is connected in the unexpanded state.

In one variation of the embodiment, the first and second ends of the radial connecting members are connected at or near the laterally adjacent peaks of radially neighboring sinusoidal members. In another variation, the first and second ends of the radial connecting members are connected to neighboring sinusoidal members between the peaks and troughs of the sinusoidal members. For example, the first and second ends of the radial connecting members may be connected to the neighboring sinusoidal members midway or approximately between the peaks and troughs of the sinusoidal members.

In one variation, the lateral offset between the points of connection of the first end and second end of each radial connecting member may be 0.5 wavelength or about 0.5 wavelength, with respect to the phase of the sinusoidal members. In another variation, the lateral offset between the points of connection of the first end and second end of each radial connecting member is 1.0 wavelength or about 1.0 wavelength, with respect to the phase of the sinusoidal members.

The sinusoidal members may be connected at a plurality of lateral positions by a “band” of radial connecting members that connect radially neighboring sinusoidal members. As shown in the drawings, the radial connecting members of a band are not directly connected to each other.

The width of the radial connecting members may be narrower than the width of the sinusoidal members, for example 25-60%, such as 25-40%, of the width of the sinusoidal members. In another variation, the sinusoidal members may have a width that narrows between the peaks and troughs of at least some of the sinusoidal members. In one embodiment of the invention, the width of the radial connecting members is in the range of about 30 to about 70 microns. For an individual radial connecting member, its width may be uniform or may vary, i.e., be non-uniform.

As used herein the term “sinusoidal” means having a succession of waves characterized by peaks and troughs, which form a periodic waveform. Sinusoidal curves include, but are not limited to, those resembling, approximating or being sine waves. As pointed out above, the longitudinal backbone members of the embodiments of the invention shown in the figures are sinusoidal. In the embodiments shown in the above referenced figures, the wave pattern of the sinusoidal backbone elements is uniform in that it does not change within sections of the prosthesis which have the backbone elements and is simple in that there are no inflection points between the peaks and troughs of the waves and no intermediate peaks or toughs between the uppermost and lowermost peaks and troughs respectively. However, the invention also provides embodiments in which there are inflection points between the peaks and troughs of the waves and/or intermediate peaks or toughs between the uppermost and lowermost peaks, but where the limitations of the various embodiments are met, such as conformation of the radial connecting members from the point of attachment to the backbone member along the member toward the lowermost trough, conforming to any intermediate inflection point and/or intermediate peaks or troughs that may be present along the path. In addition, in the embodiments shown in the above referenced figures, the amplitude of the wave pattern of each sinusoidal backbone element is the same within a section of the prosthesis having such elements. Thus, within a section of a prosthesis according to the invention that includes multiple sinusoidal backbone elements, the wave patterns of the multiple sinusoidal backbone elements may be the same.

The invention also includes embodiments wherein the central portion of the prosthesis includes at least one or more than one section having the structure and properties described for the “singular” central portion above, in which case if there is more than one of said sections, they are connected to together form a unitary central portion of the prosthesis. Such subsections of the central portion of the prosthesis may be connected to each other in any manner or by any type of structure, such as by one or more longitudinally oriented struts. The prosthesis may include an end segment at each of its ends, where each end segment includes a laterally undulating member forming a radial band comprising apices. The width of the sinusoidal members and the segments of the ends section may be the same, about the same or different. The sinusoidal members may connect to the end segments at or near the apices of the end segments.

The width of the sinusoidal members may, for example, be in the range of about 40 to about 125 microns, for example at or about 50 microns, or at or about 100 microns, or at or about 125 microns. In one embodiment, the width of the sinusoidal members is in the range of about 40 microns to about 120 microns. The width of sinusoidal members may be uniform or non-uniform. In one variation, a narrowing of width is present between at least some of the peaks and troughs of at least some of the sinusoidal members of a prosthesis according to the invention. The widths of the sinusoidal members and radial connecting elements may, respectively, both be uniform, both be non-uniform (both vary), or be such that the width of the sinusoidal members is non-uniform (varies) while the width of the radial connecting elements is uniform, or such that the width of the sinusoidal members is uniform while the width of the radial connecting elements is non-uniform (varies).

Without limitation, the following embodiments are also provided by the invention.

One embodiment of the invention provides an expandable tubular prosthesis in its unexpanded state that includes: at least one expandable at least substantially tubular portion disposed between two ends of the prosthesis, for example, a single central portion, that includes: a plurality of radially neighboring, longitudinally disposed sinusoidal members comprising peaks and troughs, the peaks and troughs of radially neighboring sinusoidal members being at least substantially in-phase with each other; and a plurality of radial connecting members that connect radially neighboring sinusoidal members, each radial connecting member having a first end and a second end and each including a plurality of segments joined by turns, wherein the first end and the second end of each radial connecting member connect to radially neighboring sinusoidal members at points of connection that are laterally offset, and wherein each radial connecting member comprises segments that at least substantially conform to the shape of the sinusoidal members to which the radial connecting member is connected.

In one variation, the first end and the second end of each radial connecting member connect to radially neighboring sinusoidal members at points of connection that are laterally offset in the range of 0.5 to 1.0 wavelength, or about 0.5 to about 1.0 wavelength, with respect to the phase of the sinusoidal members. In another variation, the degree of the offset is uniform throughout the substantially tubular portion of the prosthesis. In another variation, the width of the sinusoidal members narrows between at least substantially all of the peaks and troughs of the sinusoidal members. In another variation, the first end and the second end of each radial connecting member in the substantially tubular portion of the prosthesis respectively connect to radially neighboring sinusoidal members at points of connection at or near the laterally adjacent peaks of the radially neighboring sinusoidal members. In another variation, the first end and the second end of each radial connecting member in the substantially tubular portion of the prosthesis respectively connect to radially neighboring sinusoidal members at points of connection between the peaks and neighboring troughs for laterally adjacent peaks of the radially neighboring sinusoidal members. In another variation, each of or at least substantially all of the radial connecting members are symmetrical. It is readily seen that the radial connecting members of the embodiments shown in the figures are symmetrical. In another variation, the prosthesis comprises or consists essentially of a super-elastic metal/metal alloy. In another variation, prosthesis is self-expanding, for example, made of a shape memory metallic alloy, such as Nitinol. In another variation, all or at least at least substantially all of the radial connecting members in the substantially tubular portion laterally extends beyond the points of connection of each end thereof to the radially neighboring sinusoidal members.

A further embodiment of the invention provides a method for treating an atherosclerotic lesions, such as a vulnerable plaque, in a patient in need thereof that includes the step of deploying any of the prostheses described herein at the site of an atherosclerotic lesion, such as a vulnerable plaque lesion, in the patient. A prosthesis according to the invention may be positioned so that the central section of the prosthesis is at least partially co-extensive with the section of blood vessel that has the atherosclerotic lesion, which may be a vulnerable plaque lesion. The deployment involves an expansion of the radius of the device so that the end sections and the central section come into contact with the vessel wall. In the case of treating a vulnerable plaque, at least part of the outer surface of the prosthesis may contact the fibrous cap of the vulnerable plaque and/or at least part of the outer surface of the prosthesis may contact the vessel wall in the vicinity of the vulnerable plaque lesion. In either case, contact with the vessel wall promotes endothelialization and remodeling of at least the luminal face of a vulnerable plaque lesion. The invention also provides a general method of promoting endothelialization in a region of a blood vessel by deploying a prosthesis according to the invention in the region, irrespective of the underlying pathology of the blood vessel in the region.

The prosthesis may be delivered in a decreased radius configuration on a delivery catheter. The prosthesis may be crimped on or otherwise positioned around an inflatable deployment balloon, so that expansion of the balloon at least partially expands the prosthesis to its final working radius. For self-expanding versions of the prosthesis, use of a delivery balloon is optional. A self-expanding prosthesis may, for example, be restrained in a cylindrical cavity covered by a restraining sheath and deployed by retracting the sheath, as known in the art.

Any of the treatment methods of the invention may include a step of locating an atherosclerotic lesion, such as a vulnerable plaque lesion, to be treated by the prosthesis in a patient.

According to the invention, determining the location of a vulnerable plaque or other type of atherosclerotic lesion in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differentials characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated herein in its entirety. A method and system for detecting and localizing vulnerable plaques based on the detection of biomarkers is disclosed in U.S. Pat. No. 6,860,851, which is hereby incorporated by reference herein in its entirety. Angiography using a radiopaque and/or fluorescent dye, for example, as known in the art, may be performed before, during and/or after the step of determining the location of the vulnerable plaque, for example, to assist in positioning the prosthesis in a subject artery.

In view of the above, one embodiment of the invention provides a method of treating an atherosclerotic lesion, such as a vulnerable plaque lesion, in a blood vessel of a patient that includes the steps of: locating an atherosclerotic lesion, such as a vulnerable plaque lesion, in a blood vessel of a patient; delivering a prosthesis according to the invention in an unexpanded state to the location of the vulnerable plaque lesion in the blood vessel; and radially expanding the prosthesis to contact the wall of the blood vessel at the location of the atherosclerotic lesion. In a variation of the method, the prosthesis is deployed so that its end sections each expand to contact healthy blood vessel while the central, “working” portion of the prosthesis is expanded to cover the atherosclerotic lesion.

The prostheses of the invention may be metallic and/or polymeric in composition.

Metals used to manufacture a prosthesis according to the invention include, but are not limited to, stainless steel, titanium, titanium alloys, platinum and gold. Shape-memory metal alloys may be used to produce self-expanding versions of prostheses according to the invention. For example, suitable shape-memory alloys include, but are not limited, to Nitinol and Elgiloy.

The prostheses of the invention may, in one embodiment, be manufactured from a super elastic material, such as from a super elastic metal selected from the group of alloys consisting of copper-tin, copper-zinc, copper-zinc-aluminum, copper-zinc-tin, copper-zinc-xenon, copper-aluminum-nickel, copper-gold-zinc, gold-cadmium, gold-copper-zinc, iron beryllium (Fe3Be), iron platinum (Fe3Pt), indium-thallium, iron-manganese, iron-nickel-titanium-cobalt, nickel-titanium, nickel-titanium-vanadium, and silver-cadmium. Super elastic materials, such as Nitinol (a nickel-titanium alloy), permit the creation of highly flexible and conformable tubular prostheses/stents wherein a relatively wide-in-cross-section backbone provides the flexibility. To attain this degree of flexibility and conformability with conventional balloon expandable stent materials, such as stainless steel (SS) or Cobalt-Chromium (CoCr), would be impractical due to the very thin cross sections that would be required. Additionally, relatively wide sections that deform due to dynamic loading of an implanted prosthesis during each heart cycle would endure relatively large cyclic strains. From a durability perspective, these large cyclic strains are of less concern with super elastic materials, such as Nitinol, relative to traditional balloon expandable stent materials such as SS or CoCr.

Super elastic materials, such as Nitinol, also enable a practical mechanism for providing adequate vessel support to ensure an open lumen while minimizing and controlling the amount of stress or trauma exerted on the vessel over a relatively wide range of lumen diameters. Controlled minimization of stress over a range of diameters is possible with super elastic materials, such as Nitinol, due to their unique characteristic of having a plateau stress level whereby the stress in the material stays relatively constant over a relatively wide range of elastic strain. In contrast, conventional balloon expandable stents need to be permanently or plastically deformed in order to provide support at a given lumen diameter. This permanent deformation of the stent is induced by a balloon which is pressurized with fluid to expand to a diameter larger than the post deployment final diameter of the balloon expandable stent. Therefore, the balloon must be expanded to a larger diameter due to the elastic recoil inherent in conventional balloon expandable stents. This introduces more stress and trauma to the vessel than would be attributed to deployment of the self-expanding prostheses of the invention.

Polymers used for the manufacture of prostheses according to the invention may be biodegradable or non-biodegradable. Any suitable sorts of biodegradable polymers and/or biodegradable polymer blends may be used according to the invention. As used herein, the term “biodegradable” should be construed broadly as meaning that the polymer(s) will degrade once placed within a patient's body. Accordingly, biodegradable polymers as referred also include bioerodable and bioresorbable polymers. Suitable types of polymer material include, but are not limited to, polyester, polyanhydride, polyamide, polyurethane, polyurea, polyether, polysaccharide, polyamine, polyphosphate, polyphosphonate, polysulfonate, polysulfonamide, polyphosphazene, hydrogel, polylactide, polyglycolide, protein cell matrix, or copolymer or polymer blend thereof.

Homopolymers of polylactic acid (PLA), for example PLLA, PDLA and poly(D,L,)lactic acid, stereopolymers thereof, and copolymer of PLA with other polymeric units such as glycolide provide a number of characteristics that are useful in a polymeric prosthesis for treating a lesion of a blood vessel such as a high risk atherosclerotic plaque (vulnerable plaque). First, polymers made of these components biodegrade in vivo into harmless compounds. PLA is hydrolyzed into lactic acid in vivo. Second, these polymers are well suited to balloon-mediated expansion using a delivery catheter. Third, polymers made of these materials can be imparted with a shape-memory so that polymeric, at least partially self-expanding, tubular prostheses can be provided. Self-expanding polymeric prostheses according to the invention may also, for example, be at least partially balloon-expanded. Methods for producing biodegradable, polymeric shape-memory prostheses are described, for example, in U.S. Pat. Nos. 4,950,258, 5,163,952, and 6,281,262 each of which is incorporated by reference herein in its entirety.

Prostheses according to the invention may be manufactured by any suitable method. For example, a metallic prosthesis can be produced by laser cutting the device from a tubular blank. Methods for forming metallic tubular blanks are well known. For example, sputtering metallic material onto a mandrel may be used. In another example, the shape of the prosthesis can be laser cut or stamped out of a flat sheet of metallic material and then formed and welded into a tubular configuration. Once formed into shape, metallic prostheses according to the invention may optionally be electrochemically polished and/or etched.

The wall thickness of a prosthesis according to the invention may, for example, be in the range of about 20 microns to about 200 microns. In one embodiment, the wall thickness is equal to or less than 200 microns, for example, equal to or less than 125 microns. In one embodiment, the wall thickness is in the range of 20 microns to 125 microns. In another embodiment of the invention, the wall thickness is in the range of 20 to 60 microns. In still another embodiment, the wall thickness is in the range of 50 to 125 microns, for example in the range of 50 to 100 microns. In a further embodiment, the wall thickness is at or about 50 microns. In a further embodiment, the wall thickness is at or about 57 microns. In another embodiment, the wall thickness is at or about 100 microns. In another embodiment, the wall thickness is at or about 125 microns.

The longitudinal length of prostheses according to the invention may, for example, be in the range of 0.3 to 2.0 inch (approximately 0.8 to 5.0 cm), such as 0.5 to 1.0 inch (approximately 1.27 to 2.54 cm), such as 0.716 inch (approximately 1.819 cm).

A polymeric prosthesis according to the invention, such as one composed of polylactide, may also be laser cut from a tubular blank, such as a blank formed by extrusion molding.

Metallic or non-metallic prostheses according to the invention may optionally be coated with one or more coatings. The coating(s) may optionally include or be loaded with beneficial agents such as drugs or other compounds useful for treating vulnerable and/or for facilitating the desired functioning of the implanted prosthesis, for example, anti-thrombotic agents such as heparin to inhibit prosthesis-induced thrombosis at the treatment site. U.S. Pat. No. 5,624,411 teaches methods of coating intravascular stents with drugs, and is hereby incorporated by reference in its entirety.

Metallic or non-metallic prostheses according to the invention may optionally be provided with surface texture such as micron-scale or nanometer-scale porosity or roughness to promote desired biological results such as endothelialization. Either or both of the inner lumen surface and outer surface of a prosthesis according to the invention may be provided with texture. In one variation, at least the inner lumen surface of the prosthesis is provided with texture, to promote endothelialization. In one sub-variation only the inner lumen surface is provided with texture, to promote endothelialization. In another sub-variation, both the inner lumen surface and the outer surface of prosthesis are provided with texture, wherein the texture provided on the inner lumen surface promotes endothelialization. Texturizing techniques such as magnetron sputtering, chemical etching, electro-chemical etching, abrasive tumbling, de-alloying, abrasive media blasting, sanding, scratching, laser etching, atomic layer deposition (ALD), chemical vapor deposition (CVD) and physical vapor deposition (PVD) technologies may, for example, be used, alone or in combination. Each of U.S. Pub. Nos. 2006/0121080 and 2006/0004466 disclose surface modifications and techniques for obtaining the same that may be employed with the prostheses of the invention, and is incorporated by reference herein in its entirety.

Each of the patents and other documents cited herein is hereby incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A radially expandable tubular prosthesis in its unexpanded state, comprising:
an expandable at least substantially tubular central portion disposed between two ends of the prosthesis, comprising:
a plurality of radially neighboring, longitudinally disposed sinusoidal members comprising peaks and troughs, the peaks and troughs of radially neighboring sinusoidal members being at least substantially in-phase with each other, so that laterally adjacent, oppositely facing peaks of radially neighboring sinusoidal members are offset by 0.5 wavelength or about 0.5 wavelength with respect to the phase of the sinusoidal members, and wherein the width of the sinusoidal members narrows between at least substantially all of the peaks and troughs of the sinusoidal members; and
a plurality of radial connecting members that connect radially neighboring sinusoidal members, each radial connecting member having a first end and a second end and each comprising a plurality of segments and turns,
wherein the first end and the second end of each radial connecting member respectively connect to radially neighboring sinusoidal members at points of connection at or near the laterally adjacent peaks of the radially neighboring sinusoidal members,
wherein each radial connecting member comprises segments that conform to the troughs of the sinusoidal members adjacent to the points of connection of the ends of the radial connecting member, said troughs being opposite the peaks of the radially neighboring sinusoidal members at or near which the ends of the radial connecting member are connected, and
wherein each radial connecting member laterally extends beyond the points of connection of each end thereof at or near laterally adjacent peaks of radially neighboring sinusoidal members.

2. The prosthesis of claim 1, wherein the sinusoidal members are connected at a plurality of lateral positions by a band of radial connecting members connecting radially neighboring sinusoidal members.

3. The prosthesis of claim 1, further comprising:
a radially expandable end segment at each end of the central portion of the prosthesis, each end segment comprising a laterally undulating member forming a radial band comprising inner turn portions facing the central portion of the prosthesis and outer turn portions at each end of the prosthesis,
wherein each of the ends of the sinusoidal members are connected to an end segment.

4. The prosthesis of claim 3, wherein at both ends of the prosthesis a radiopaque marker is provided between at least some of the ends of the sinusoidal members and the end segments to which they are connected.

5. The prosthesis of claim 3, wherein a structure defining an opening for receiving a radiopaque marker material is provided between at least some of the ends of the sinusoidal members and the end segments to which they are connected.

6. The prosthesis of claim 5, wherein the structure defining an opening for receiving a radiopaque marker material is provided with a radiopaque marker material.

7. The prosthesis of claim 5, wherein a hinge structure is provided at the junction of the structure defining an opening for receiving a radiopaque marker material and the end segment to which it connects.

8. The prosthesis of claim 3, wherein
the inner turn portions of each of the end segments are alternately connected to the sinusoidal members and to laterally adjacent radial connecting members;
a structure defining an opening for receiving a radiopaque marker material is provided between each of the ends of the sinusoidal members and the inner turn portion of end segment to which it connects; and
the prosthesis comprises connecting struts that connect the inner turn portions that are connected to laterally adjacent radial connecting members.

9. The prosthesis of claim 8, wherein a hinge structure is provided at the junction of the structure defining an opening for receiving a radiopaque marker material and the turn portion of the end segment to which it connects.

10. The prosthesis of claim 3, wherein
the inner turn portions of each of the end segments are alternately connected to the sinusoidal members and to laterally adjacent radial connecting members;
a radiopaque marker is provided between each of the ends of the sinusoidal members and the inner turn portion of end segment to which it connects; and
the prosthesis comprises connecting struts that connect the inner turn portions that are connected to laterally adjacent radial connecting members.

11. The prosthesis of claim 1, wherein the prosthesis has a wall thickness in the range of about 50 to about 100 microns.

12. The prosthesis of claim 11, wherein the prosthesis has a wall thickness of about 57 microns.

13. The prosthesis of claim 1, wherein the width of the sinusoidal members is in the range of about 40 microns to about 120 microns.

14. The prosthesis of claim 8, wherein the width of the radial connecting members is in the range of about 30 to about 70 microns.

15. The prosthesis of claim 1, wherein the maximum width of the radial connecting members is less than the maximum width of the sinusoidal members.

16. The prosthesis of claim 1, wherein the prosthesis is self-expanding.

17. The prosthesis of claim 1, wherein the prosthesis is composed of a super elastic metallic alloy.

18. A radially expandable tubular prosthesis in its unexpanded state, comprising:
at least one radially expandable at least substantially tubular portion disposed between two ends of the prosthesis, comprising:
a plurality of radially neighboring, longitudinally disposed sinusoidal members comprising peaks and troughs, the peaks and troughs of radially neighboring sinusoidal members being at least substantially in-phase with each other, so that laterally adjacent, oppositely facing peaks of radially neighboring sinusoidal members are offset by 0.5 wavelength or about 0.5 wavelength with respect to the phase of the sinusoidal members, and wherein the width of the sinusoidal members narrows between at least substantially all of the peaks and troughs of the sinusoidal members; and
a plurality of radial connecting members that connect radially neighboring sinusoidal members, each radial connecting member having a first end and a second end and each comprising a plurality of segments and turns,
wherein the first end and the second end of each radial connecting member respectively connect to radially neighboring sinusoidal members at points of connection at or near the laterally adjacent peaks of the radially neighboring sinusoidal members,
wherein each radial connecting member comprises segments that conform to the troughs of the sinusoidal members adjacent to the points of connection of the ends of the radial connecting member, said troughs being opposite the peaks of the radially neighboring sinusoidal members at or near which the ends of the radial connecting member are connected, and
wherein each radial connecting member laterally extends beyond the points of connection of each end thereof at or near laterally adjacent peaks of radially neighboring sinusoidal members.

19. The prosthesis of claim 18, wherein the prosthesis is self-expanding.

20. A radially expandable tubular prosthesis in its unexpanded state, comprising:
at least one expandable at least substantially tubular portion disposed between two ends of the prosthesis, said portion composed of a super elastic metal alloy, having a wall thickness in the range of 40 to 100 microns and comprising:
a plurality of radially neighboring, longitudinally disposed sinusoidal members comprising peaks and troughs, the peaks and troughs of radially neighboring sinusoidal members being at least substantially in-phase with each other, so that laterally adjacent, oppositely facing peaks of radially neighboring sinusoidal members are offset with respect to the phase of the sinusoidal members; and
a plurality of radial connecting members that connect radially neighboring sinusoidal members, each radial connecting member having a first end and a second end and each comprising a plurality of segments and turns,
wherein the first end and the second end of each radial connecting member respectively connect to radially neighboring sinusoidal members at points of connection at or near the laterally adjacent peaks of the radially neighboring sinusoidal members,
wherein each radial connecting member comprises segments that conform to the troughs of the sinusoidal members adjacent to the points of connection of the ends of the radial connecting member, said troughs being opposite the peaks of the radially neighboring sinusoidal members at or near which the ends of the radial connecting member are connected, and
wherein each radial connecting member laterally extends beyond the points of connection of each end thereof at or near laterally adjacent peaks of radially neighboring sinusoidal members.
